(19) United States
(12) Patent Application Publication
(10) Pub. No.: US 2023/0204556 A1
(43) Pub. Date: Jun. 29, 2023

(54) A METHOD, A SENSING MODULE AND A KIT TO DETECT A CHEMICAL SUBSTANCE IN AN ENVIRONMENT

(71) Applicant: PHILIP MORRIS PRODUCTS S.A., Neuchâtel (CH)

(72) Inventors: Rui Nuno BATISTA, Morges (CH); Ricardo CALI, Mannheim (DE); Tobias LIPP, Santiago de Compostela (ES)

(73) Assignee: PHILIP MORRIS PRODUCTS S.A., Neuchâtel (CH)

(21) Appl. No.: 17/923,697

(22) PCT Filed: Apr. 15, 2021
(86) PCT No.: PCT/EP2021/059804

§ 371 Date: Nov. 7, 2022

(30) Foreign Application Priority Data
May 7, 2020 (EP) 20173541.2

(51) Int. Cl. G01N 33/00

(52) U.S. Cl. CPC G01N 33/0065; G01N 33/004

(57) ABSTRACT

The present invention relates to A method to detect a specific chemical substance in an environment, the method comprising:—providing a sensing layer adapted to sense the specific chemical substance;—providing a substrate layer, the substrate layer comprising a single-use wireless transmitter;—attaching the sensing layer to the substrate layer so as to form a sensing module;—detecting the amount of the specific chemical substance in the environment by means of the sensing layer;—if the amount of the specific chemical substance is above or below a first threshold, sending data representative of the sensed specific chemical substance to a receiving device using the wireless transmitter. The invention also relates to a sensing module and a kit to measure the specific chemical substance.

The present invention relates to a method, a sensing module and a kit to detect a specific chemical substance in an environment, the method and the kit including a single-use sensing module.

Limiting exposure to certain chemical substance may have an impact on a person's health. There are some chemical substances which are potentially harmful for the majority of living beings and others which can be dangerous for only some individuals, for example allergens which can cause allergic reactions in predisposed persons. Individuals sensitive to specific pollutants like dust, volatile organic compounds or pollen may have such an allergic reaction when they enter an unknown environment. Leaving the familiar surrounding can thus be problematic for persons with allergies or respiratory problems if they cannot monitor the ambient air around them.

Known air quality monitoring systems are generally fixedly mounted in a room. Further, in order to function, they need a source of energy and for this reason they are commonly connected to the mains. Portable air monitoring products which allow users to analyse air quality while moving from one place to another are also known. These devices still have to be charged and are generally rather bulky.

Therefore, there is the need to have a method, a sensing module and a kit to detect air quality in an environment, for example to detect the presence of a specific chemical substance, which allow portability. The method, the sensing module and the kit are preferably accurate and capable of alerting a user of poor air quality, for example due to the presence of a specific chemical substance, at any location. Further, the method, sensing module and kit are preferably relatively cheap.

According to an aspect, the present invention relates to a method to detect a specific chemical substance in an environment. The method may comprise providing a sensing layer adapted to sense the specific chemical substance. The method may also comprise providing a substrate layer, the substrate layer comprising a single—use wireless transmitter. The method may also comprise attaching the sensing layer to the substrate layer so as to form a sensing module. The method may also comprise sensing the amount of the specific chemical substance in the environment by means of the sensing layer. Further, the method may comprise, if the amount of the specific chemical substance is above or below a first threshold, sending data representative of the sensed specific chemical substances to a receiving device using the wireless transmitter.

In the method of the invention, sensing of a specific chemical substance is performed by means of a sensing module. Due to the layered structure of the sensing module, easy transport of the same may be feasible. Further, according to the invention, the sensing module is a single use sensing module. The sensing module is adapted to send a single signal. This advantageously allows the reduction of the necessary electronics on the sensing module, allowing to realize low cost but still accurate measurements.

The sensing module comprises a sensing layer. The sensing layer is adapted to sense a specific chemical substance. The specific chemical substance may be a gas, such as CO or CO₂, volatile organic compounds, dust, any allergens or a combination thereof. The sensing layer may be adapted to detect the presence of the specific chemical substance in the environment, for example in the air present in the environment. In case of detection, the sensing layer's response may be either that the specific chemical substance is “present” or that it is “not present”. In this case, the sensing of the sensing layer is substantially digital, either “present” or “absent”. The sensing layer may be adapted to measure the amount of the specific chemical substance in the environment. The sensing layer may measure for example the concentration of the specific chemical substance in the air present in the environment. In this case, the sensing of the sensing layer may be considered analog sensing. The sensing layer may be configured to be capable of switching from a configuration in which it only detects the absence or presence of the specific chemical substance to a configuration where it measures the concentration of the specific chemical substance, or vice-versa.

The sensing layer preferably comprises a sensor. The type of sensor comprised in the sensing layer depends on the specific chemical substance to be sensed. The sensor module may include a dust level sensor, CO₂ concentration sensor, VOC (volatile organic compound) concentration sensor, or others. The sensing layer further preferably includes a sheet of material on which the sensor is fixed. The sensor may be formed on the sheet of material for example by means of ink printing, vapour deposition, or sputtering. The sheet of material is preferably a plastic sheet. Preferably, it comprises a polymeric sheet.

The sensor may include a semiconductor metal oxide gas sensor. The sensor may include a carbon nanotube-based gas sensor.

Preferably, the thickness of the sensing layer, at its thickest portion, is comprised between 1 millimetre and 6 millimetres. Preferably, the maximum dimensions of the sensing layer in the two directions perpendicular to the direction defined by the thickness are comprised between 10 millimetres and 100 millimetres, more preferably between 20 millimetres and 50 millimetres.

In addition to the sensor to sense the specific chemical substance, the sensing layer may include other sensors. For example, the sensing layer may include an electromagnetic sensor, a biometric sensor, a temperature sensor, a liquid sensor or a combination thereof.

Further, the sensing layer may include a single sensor to sense the specific chemical substance. The sensing layer may include more than one sensor to sense the same specific chemical substance. Further, the sensing layer may include more than one sensor to sense different specific chemical substances.

The sensing module further comprises a substrate layer. The substrate layer preferably includes a wireless transmitter. The wireless transmitter is adapted to send data representative of the sensing performed by the sensing layer. The wireless transmitter preferably comprises an antenna to send the data. Preferably, the wireless transmitter sends an electromagnetic signal carrying information related to the sensed specific chemical substance. The wireless transmitter is a “single—use” wireless transmitter. Single—use means that the wireless transmitter is designed to be used once. The “use” of the wireless transmitter is the sending of data. The wireless transmitter is configured to send data representative of the sensing performed by the sensing layer once. Then, the wireless transmitter may be discarded. A single—use transmitter is a transmitter that transmit data representative of the sensing performed by the sensing layer only once. The data are preferably complete, that is, the wireless transmitter transmits only once the complete data relative to one sensing (for example, the wireless transmitter transmits the complete data of one measurement). The wireless transmitter does not sent further signals after the transmission of the data relative to a sensing. Thus, once the wireless transmitter is used for sending the first signal, it is not used anymore. This can be obtained for example connecting the wireless transmitter to an energy source. Preferably, the energy source has stored an amount of energy equal or bigger than the energy required to send by the wireless transmitter data representative of the sensing performed by the sensing layer once. The energy source has stored less energy than the energy required to send by the wireless transmitter data representative of the sensing performed by the sensing layer twice. In the following, with “signal” sent by the wireless transmitter, the data representative of the sensing performed by the sensing layer are meant.

The wireless transmitter may include a transponder.

The sensing layer is attached to the substrate layer. For example, the sensing layer, or the substrate layer, may comprise an adhesive, in order to glue the sensing layer and the substrate layer together. Any other means to attach the sensing layer and the substrate layer together can be used as well, like for example, micro-suction or electrostatic. Preferably, the attachment between the sensing layer and the substrate layer is such that an electrical connection between the sensing layer and the substrate layer is established. An electrical connection between the sensing layer and the substrate layer is a connection that allows an electrical signal to travel from the substrate layer to the sensing layer or vice-versa.

Preferably, the thickness of the substrate layer, at its thickest portion, is comprised between 1 millimetre and 6 millimetres. Preferably, the maximum dimensions of the substrate layer in the two directions perpendicular to the direction defined by the thickness are comprised between 10 millimetres and 100 millimetres, more preferably between 20 millimetres and 50 millimetres.

Preferably, the sensing layer defines a first surface and a second surface, and the substrate layer is attached to the first surface of the sensing layer. Preferably, the substrate layer defines a first surface and a second surface, and the second surface of the substrate layer is attached to the first surface of the sensing layer. Preferably, the area of the first surface of the sensing layer is substantially identical to the area of the second surface of the substrate layer in shape. Preferably, the area of the first surface of the sensing layer is substantially identical to the area of the second surface of the substrate layer, in dimension. Preferably, the first surface of the sensing layer and the second surface of the substrate layer are congruent, coinciding substantially exactly when superimposed. Preferably, between the first surface of the sensing layer and the second surface of the substrate layer, an adhesive layer is interposed.

Further, the method may comprise, if the amount of the specific chemical substance is above or below a first threshold, sending data representative of the sensed chemical substances to a receiving device using the wireless transmitter. Preferably, the sending of data takes place only once. Once the wireless transmitter has sent the data representative of the sensed chemical substance for the first time, the wireless transmitter does not send data any more.

The “amount” of specific chemical substance may be either a measured amount, or a “present” or “absent” indication. Therefore, “the amount of the specific chemical substance is above or below a first threshold” may mean that the measured quantity (for example, concentration) MQ of the specific chemical substance in the air of the environment is above or below a first threshold FT:

### MQ>FT or MQ<FT.

“The amount of the specific chemical substance is above or below a first threshold” may also mean that the specific chemical substance is present (above threshold) or absent (below threshold).

For example, a user has an allergy to a specific allergen. The sensing module may sense the concentration of the specific allergen in the air and, in case the concentration is higher than a first threshold, the sensing module may send the data representative to the sensed allergen to the receiving device. The receiving device may be for example a mobile phone. The receiving device may alert the user that the allergen is present in the environment at a relatively “high” concentration. Alternatively to measuring the amount, the sensing module may send data relative to the sensed allergen to the receiving device as soon as any presence of such allergen is sensed in the environment, regardless of the concentration of the allergen.

The sensing module may sense the quantity of a specific gas present in a room. In case the gas is oxygen, the sensing device may send data relative to the sensed oxygen as soon as the concentration of oxygen in the environment falls below a first threshold, because a too low oxygen quantity may be harmful to humans.

Preferably, the first threshold is tunable. The method may include the step of tuning the first threshold. The first threshold may be tuned depending on the users' needs, for example depending on their allergy sensitivity. Thus, the first threshold can be raised or lowered on demand. The first threshold may be set or modified using the receiving device. A user may input the desired first threshold, or may modify the already set first threshold, inputting a new value in the receiving device. The receiving device may send a signal to the sensing layer to set the first threshold based on the input entered in the receiving device.

The fact that the specific chemical substance is “absent” in the environment means that the substance has a concentration so low that it is not detectable by the sensing layer, and thus “absent” may mean that there are in reality traces of the specific chemical substance, in an amount that depends on the sensitivity or resolution of the selected sensing layer.

If the amount of specific chemical substance as detected by the sensing layer is above or below the first threshold, data which represent the sensed specific chemical substance are sent from the sensing module, using the wireless transmitter, to a receiving device. After the data have been sent, the wireless transmitter cannot be used again.

The receiving device is preferably external from the sensing module. The receiving device may be any type of device configured to operate or communicate in a wireless environment. By way of example, the receiving device may be configured to transmit or receive wireless signals, and may include a user equipment, a mobile station, a fixed or mobile subscriber unit, a pager, a cellular telephone, a personal digital assistant (“PDA”), a Smartphone, an iPhone, a laptop, a netbook, a personal computer, a wireless sensor, consumer electronics, and other transmitter/receivers known in the art. The receiving device can be connected to a human or also to a machine.

The data sent by the wireless transmitter may contain the information of the amount of specific chemical substance, or of the presence or absence of the specific chemical substance.

Preferably, the receiving device includes a receiver adapted to receive the data sent by the wireless transmitter. Preferably, the receiving device includes a control unit adapted to elaborate the received data. Preferably, the receiving device includes a display to visualize the received data or the elaborated received data.

In order to send data by the wireless transmitter, the wireless transmitter is preferably activated. “Activate” the wireless transmitter means to make it function, and the function of the wireless transmitter is to send a signal. Therefore, the activation of the wireless transmitter causes the sending of a signal by the wireless transmitter. The wireless transmitter is configured so that it sends as signals data representative of the sensed specific chemical substance. In this case, “signal” means the data representative of the sensing performed by the sensing layer. In order to activate the wireless transmitter, preferably the sensing module comprises an energy source. Preferably, the energy source is a stand-alone energy source. A stand-alone energy source can function independently of other hardware. In this case, it means that the energy source present in the sensing module can produce enough energy for the transmission of the data relative to the sensed chemical substance by the wireless transmitter without using an additional energy source external to the sensing module.

Preferably, the energy source is comprised in the substrate layer. Preferably, the energy source is integrated in the substrate layer. The energy source may also comprise an energy storage unit adapted to be charged by the energy source. The energy source may consist of the energy storage unit only, which function, when charged, as an energy source. The energy source may be used to charge the energy storage unit. Preferably, the energy source is adapted to charge the energy storage unit once. Preferably, the energy storage unit is not rechargeable.

The energy source may be for example a piezoelectric device, a solar panel, a thermoelectric or Seeback generator. The energy source preferably generates an electric signal, for example, a current. The electric energy generated by the energy source may be stored in an energy storage unit.

The energy source or the energy storage unit may generate a voltage comprised between 1 Volt and 5 Volt, more preferably between 1.5 Volt and 2 Volt. The energy source or the energy storage unit may generate an electrical current comprised between 50 nanoampere and 500 nanoampere, more preferably between 150 nanoampere and 250 nanoampere.

In order to activate the wireless transmitter, the energy produced by the energy source or the energy stored in the energy storage unit is preferably sent to the wireless transmitter so that the wireless transmitter is capable of sending the data relative to the sensed specific chemical substance to the receiving device.

After the data relative to the sensed specific chemical substance have been sent to the receiving device by the wireless antenna, in case the first threshold has been reached, the wireless antenna is not re-used. According to the invention, the sensing module is a single-use sensing module, so that, after a signal containing the data of the sensed chemical substance has been sent, it is not used anymore. Only one signal concerning one single measurement is sent.

Preferably, after the sending of the data relative to the sensed specific chemical substance, the energy source, for example the energy storage unit, is substantially depleted. Preferably, no more energy can be produced to generate another energy amount necessary to activate again the wireless transmitter and send other data relative to a sensed specific chemical substance to the receiving device. Thus, the wireless transmitter can be activated only once to send data relative for example to a single measurement. For example, the energy source includes a battery containing an amount of energy enough to activate the wireless transmitter once, but not twice. More preferably, the battery is not rechargeable. The wireless transmitter therefore cannot send another signal in form of data because there is not enough energy available to activate it again and allow the transmission. The energy storage unit may be not completely depleted, but it may still contain some energy. This remaining energy is however not enough for a second activation of the wireless transmitter.

The fact that the sensing module is single-use, allows the realization of a relatively cheap sensing module. The configuration of the sensing module, having two layers (the sensing layer and the substrate layer) attached to each other, allows the realization of a sensing module which is easily transportable, for example attached to clothes or accessories, due to its contained dimensions. The layers configuration forms a sensing module which is relatively thin and discreet. However, the wireless transmitter in the sensing module still needs to be activated in order to send a signal relative to the sensed chemical substance to the receiving device. This activation can be done by means of an energy source. However, the energy source—if present—needs to be relatively “small” as well, otherwise it would not fit within the layered sensing module. The fact that the sensing module has exhausted its use after a first signal is sent (that is, after the data relative to the sensed specific chemical substance are sent once) allows to dimension the energy source for producing an amount of energy just enough to activate the wireless transmitter only once. The requirement of a “small” energy amount in turn allows to select a relatively small and cheap energy source. Furthermore, as the sensing module needs to function only until the data are sent, the various components forming the sensing module can also have a relatively short life span. The method of the invention can be thus performed by a relatively small and cheap sensing module.

The method may comprise discarding the sensing module after sending the data representative of the sensed specific chemical substance for the first time. Because the sensing module cannot be used any more after the first signal of the sensed specific chemical substance is sent, it can be discarded.

Preferably, the method includes: setting a maximum life span of the sensing module. Preferably, the maximum life span of the sensing module is set as the earliest of: the expiry of a predetermined time interval, and sending the data representative of the sensed specific chemical substance for the first time. More preferably, the method includes discarding the sensing module when the maximum life span of the sensing module has been reached. It may happen that the sensing module does not send data relative of a chemical substance to the receiving device for some time. For example, the amount of the chemical substance may stay for a relatively long while above (or below) the first threshold and no activation of the wireless transmitter takes place. However, as mentioned, the sensing device is preferably a “cheap” device and the functioning of all its components may not be guaranteed after a predetermined time interval. Therefore, the sensing module may be discarded also when the sensing module lasts for a time equal to or longer than a predetermined time interval. The predetermined time interval may be set using the receiving device. The sensing module may thus be discarded when the predetermined time interval has elapsed, if this takes place before the data relative to the sensed specific chemical substance are sent by the wireless transmitter. The maximum life span can be selected as the “first two expire” between two intervals, the predetermined time interval and a time interval that ends when the wireless transmitter sends the data relative to the sensed specific chemical substance. The maximum life span is preferably determined by the earliest of these moments: the expiry of the predetermined time interval and the moment in which the wireless transmitter sends the data relative to the sensed specific chemical substance.

Preferably, the method comprises: providing the sensing module with an energy source. In order to activate the wireless transmitter so that the wireless transmitter has the necessary energy to send the data relative to the sensed specific chemical substance, preferably the sensing module comprises an energy source. Preferably, the energy source is integrated in the substrate layer. Preferably, the energy source is a stand-alone energy source. Preferably, the energy source is adapted to provide an amount of energy enough for the step of sending the data of the sensed specific chemical substance by the wireless transmitter. Preferably, the energy source is adapted to provide an amount of energy enough for the step of sending the data of the sensed specific chemical substance by the wireless transmitter only once.

Preferably, the step of providing a substrate layer includes: providing a flexible substrate layer. Preferably, the step of providing a sensing layer includes providing a flexible sensing layer. Preferably, a sensing module having a flexible substrate layer, or a flexible sensing layer, or preferably both, is easily attachable to several items, such as clothes. In this way, it may be easily transported wherever needed.

More preferably, the method comprises the step of generating energy by mechanical deformation of the flexible substrate layer in order to activate the wireless transmitter. Even more preferably, the method includes supplying the generated energy to the wireless transmitter. The flexibility of a flexible substrate layer may be used as an energy generator. The deformations of the substrate layer may be used to generate energy using a physical effect, such as for example a piezoelectric effect. The energy generated by the deformations may be used to activate the wireless transmitter and to send the data relative to the sensed specific chemical substance. The energy generated by the mechanical deformation may be stored before being used.

Preferably, the step of providing a sensing layers includes: covering the sensing layer with a protective layer. Preferably, the method includes: removing the protective layer from the sensing layer before the step of sensing the specific chemical substance in the environment. When the sensing module is used according to the method of the invention, it is desirable that the sensing of the specific chemical substance takes place when a user desires it, that is, when the user for example reaches a new environment. Measurements of chemical substances taking place when the sensing module is stored or in random environments are generally not useful for a user. Therefore, it is preferred to apply a protective layer to the sensing layer of the sensing module. The protective layer has preferably a twofold action. First, the protective layer may protect the sensing layer, and preferably the sensors, from damages. Damages may occur if the sensing layer scratches or hits other objects, for example. Furthermore, the protecting layer may prevent the sensing layer from performing the action of sensing. Therefore, while the sensing layer is covered by the protective layer, the sensing layer cannot sense the specific chemical substance. The predetermined time interval of the sensing module preferably starts when the protective layer is removed from the sensing layer.

Preferably, the method includes setting a predetermined time interval of the sensing module and beginning the predetermined time interval of the sensing module from the moment in which the protective layer is removed from the sensing module. The predetermined time interval is used to calculate the maximum life span of the sensing module. Thus, the removal of the protective layer represents the beginning of the predetermined time interval. The predetermined time interval may be used to calculate the life span of the sensing module.

Preferably, the method comprises: setting a first threshold of the specific chemical substance; or modify a pre-set first threshold of the specific chemical substance in the sensing layer. The wireless transmitter sends data relative to the sensed specific chemical substance to the receiving device if the amount of sensed chemical substance is above or below a first threshold. This first threshold may be fixed, that is, set once and then not changeable, or it may be tuned according to the user's needs. Further, the first threshold may be set when the sensing module is fabricated, for example in the factory, or it may be set by the user.

Preferably, the method comprises: providing the sensing module with a non-rechargeable energy storage unit. Preferably, the method further comprises depleting the non-rechargeable energy storage unit by sending data representative of the specific chemical substance to the receiving device using the wireless transmitter. The sensing module may comprise a non-rechargeable energy storage unit, which means an energy storage unit that, after its depletion, cannot (or it is supposed not to) be recharged. Preferably, the sensing module comprises an energy source and a non-rechargeable energy unit, the energy source being adapted to charge the non-rechargeable energy unit once. The non-rechargeable energy unit is then preferably electrically connected to the wireless transmitter to activate the same when the data relative to the sensed chemical substance are to be sent. For example, the non-rechargeable energy unit may include a capacitor. The capacitor may include three capacitor layers. The three capacitor layers may include: a first conductive layer and a second conductive layer separated by a dielectric layer. Material for these layers are known in the art. Preferably, the energy source includes a piezoelectric device. Preferably, the piezoelectric device includes a first electrode layer and a second electrode layer. Preferably, the piezoelectric device includes a piezoelectric nanocomposite layer sandwiched between the first electrode layer and the second electrode layer. Preferably, the piezoelectric nanocomposite layer comprises a composite of one or more of BaTiO₃, ZnO, MoS₂ or WSe₂

Preferably, the method includes generating a warning signal when the data representative of the sensed specific chemical substance are received by the receiving device. When the data relative to the sensed specific chemical substance reaches the receiving device, it means that the amount of chemical substance has exceeded or it is lower than the first threshold. This in turn may mean that there is a potential hazard for the user in the environment where the sensing module is located. In order to warn the user of this potentiality, a warning signal may be issued. For example, the warning signal may be issued by the receiving device. The warning signal may be an acoustic signal, for example an acoustic loud sound, such as an alarm. The warning signal may be a visual signal, for example a blinking light of a specific colour. The warning signal may be a video-acoustic signal. Furthermore, the warning signal may comprise vibrations of the receiving device. If the warning signal is not blocked after a set time interval, the method may include the step of calling a selected phone number or IP address by the receiving device.

Preferably, the method comprises: providing a set of sensing layers, each sensing layer of the set adapted to sense a specific chemical substance different from the specific chemical substance sensed by another sensing layer of the set. Preferably, the method comprises selecting a specific chemical substance to be sensed. Preferably, the method comprises selecting, from the set of sensing layers, a sensing layer for the selected specific chemical substance. The method of the invention may be used to sense more than a chemical substance. In order to sense a plurality of specific chemical substances, and to send data representative of each sensed specific chemical substance, if the sensed specific chemical substance is above or below a threshold, a set of sensing layers is provided. Each sensing layer of the set is adapted to sense a specific chemical substance, preferably different from the specific chemical substances sensed by the other sensing layers of the set. Preferably, for each sensing layer of the set, a threshold is set. For example, if the set includes N sensing layers, N thresholds are set. If the amount of specific chemical substance sensed by the i-th sensing layer of the set of N sensing layers is above or below the i-th threshold, then the wireless transmitter send data relative to the specific chemical substance sensed by the i-th sensing layer. Preferably, some of the N thresholds are tunable. Preferably, all thresholds of the N thresholds are tunable. In this way an optimized sensing module for the users' needs may be formed.

Preferably, the step of providing a set of sensing layers includes: providing each sensing layer of the set with a machine readable code identifying the specific chemical substance sensed by the sensing layer. In order to easily identify which specific chemical substance is sensed by which sensing layer in a set, or to easily identify the sensing layers, some sensing layers of the set includes a machine readable code. Probably, all sensing layers of the set include a machine readable code. The machine readable indicia may comprise a bar code. The bar code may include a linear or one-dimensional (1D) bar code. Further, the bar code may include geometric patterns, called matrix codes or two-dimensional (2D) bar codes. The bar code represents data about the specific chemical substance sensed by the sensing layer which carries the bar code.

The step of providing a set of sensing layers may include: providing each sensing layer of the set with a human readable code identifying the specific chemical substance sensed by the sensing layer. The name of the specific chemical substance sensed by the sensing layer may be written on the sensing layer.

More preferably, the method comprises scanning the machine readable indicia by means of the receiving device. Preferably, the one dimensional bar codes may be read by optical scanners, called barcode readers. Two-dimensional bar codes can be read or deconstructed using application software on receiving devices including a camera. An example of a receiving device having this property is for example a smartphone.

Preferably, the method comprises selecting at least two sensing layers from the set of sensing layers; and attaching the at least two sensing layer to the same substrate layer. The sensing module may sense several different specific chemical substances using the same resources, such as for example the same wireless transmitter, or the same energy source or the same non-rechargeable energy storage unit. In this way, the sensing module has relatively small dimensions and it is relatively cheap.

According to another aspect, the invention relates to a sensing module for detecting a specific chemical substance in an environment, comprising a sensing layer adapted to sense the specific chemical substance. The sensing module may further comprise a substrate layer attached to the sensing layer. The substrate layer may comprise: a wireless transmitter. The substrate layer may comprise a non-rechargeable energy storage unit electrically connectable to the wireless transmitter and adapted to contain an amount of energy sufficient to allow a single activation of the wireless transmitter. The sensing module may also comprise a switch adapted to activate the wireless transmitter by connecting it to the non-rechargeable energy storage unit if the amount of the specific chemical substance sensed by the sensing layer is above or below a first threshold, so that the wireless transmitter sends data representative of the specific chemical substances.

According to another aspect, the invention relates to a kit for detecting a specific chemical substance in an environment. The kit comprises: a set of sensing layers attached to a common base, each sensing layer adapted to sense a specific chemical substance. The kit may also comprise a substrate layer, the substrate layer being adapted to be attached to at least one of the sensing layers of the set so as to form a sensing module. The substrate layer may comprise: a wireless transmitter. The substrate layer may comprise a non-rechargeable energy storage unit electrically connectable to the wireless transmitter and adapted to contain an amount of energy sufficient to allow a single activation of the wireless transmitter. The sensing module may also comprise a switch adapted to activate the wireless transmitter by connecting it to the non-rechargeable energy storage unit if the amount of the specific chemical substance sensed by the sensing layer is above or below a first threshold, so that the wireless transmitter sends data representative of the sensed specific chemical substance. The kit may also comprise a receiving device adapted to receive the data sent by the wireless transmitter.

The characteristics and advantages of the sensing module and of the kit have been already detailed with reference to the description of the method of the invention and are not herewith repeated.

Preferably, the wireless transmitter comprises a transponder. Preferably, the wireless transmitter comprises an antenna. Preferably, the transponder comprises a transponder chip.

Preferably, the substrate layer comprises an activation layer including the wireless transmitter, and an energy layer comprising the non-rechargeable energy storage unit, the activation layer and energy layer being attached one to the other and electrically connected. Preferably, the substrate layer is multi-layered. Each layer of the multi-layered substrate layer has a specific function. The energy layer preferably includes the non-rechargeable energy storage unit to furnish energy to the wireless transmitter. The activation layer preferably includes the wireless transmitter to send data relative to the sensed chemical substance. A separation of functions in different layers may lead to a simplified fabrication of the sensing module.

Preferably, the non-rechargeable energy storage unit comprises a capacitor to store energy. Preferably, the capacitor comprises a three layers structure.

Preferably, the sensing layer comprises: a protective layers covering the sensing layer.

Preferably, the substrate layer comprises an energy source. Preferably, the energy layer includes the energy source. Preferably, the energy source is integrated in the energy layer. Preferably, the energy source is adapted to charge the non-rechargeable energy storage unit.

Preferably, the energy source comprises a piezoelectric device.

Preferably, each sensing layer of the set comprises a machine readable code including information on the specific chemical substance sensed by the sensing layer. Preferably, each sensing layer of the set comprises a human readable code including information on the specific chemical substance sensed by the sensing layer.

A “energy storage unit” is a unit adapted to store energy. Energy storage is the capture of energy produced at one time for use at a later time. A device that stores energy may be for example a battery. Energy may come in multiple forms, however in the present context the stored energy is preferably electromagnetic energy or electrochemical energy. A “not rechargeable” energy storage unit is an energy storage unit that is designed to be used once and discarded. The not rechargeable energy storage unit is not supposed to be recharged, for example with electricity, and reused.

A “wireless transmitter” is an electronic device which produces electromagnetic waves carrying a signal with an antenna. The transmitter itself may generate a radio frequency alternating current, which is applied to the antenna. When excited by this alternating current, the antenna radiates electromagnetic waves, such as radio waves.

A “layer” in the present context defines a sheet of material having a thickness smaller than its other two dimensions by at least a factor of 10. Preferably, the other two dimensions are one order of magnitude larger than the thickness of the sheet.

“Flexible” in the present context denotes the ability of a layer to change shape without breaking when a force is applied.

The word “sense” a chemical substance means either detect, or measure, or both, a chemical substance. Therefore “sensing” generally defines the activity of a sensor with respect to a chemical substance, encompassing the cases in which the output of the sensor in the step of sensing is digital or analogue.

“Chemical substance” in the following means any material with a definite chemical composition, for example chemical element, chemical compounds, or alloys. It may include molecules.

In the present text, the verbs “comprise” and “include” are synonyms and they both indicate a non-exhaustive list of features. The verb “consist” indicates an exhaustive list.

The invention is defined in the claims. However, below there is provided a non-exhaustive list of non-limiting examples. Any one or more of the features of these examples may be combined with any one or more features of another example, embodiment, or aspect described therein.

Example 1 Ex1: A method to detect a specific chemical substance in an environment, the method comprising:
providing a sensing layer adapted to sense the specific chemical substance;
providing a substrate layer, the substrate layer comprising a single-use wireless transmitter;
attaching the sensing layer to the substrate layer so as to form a sensing module;
sensing the amount of the specific chemical substance in the environment by means of the sensing layer;
if the amount of the specific chemical substance is above or below a first threshold, sending data representative of the sensed specific chemical substance to a receiving device using the wireless transmitter.

Example Ex2: The method according to Ex1, comprising the step of:
discarding the sensing module after sending the data representative of the sensed specific chemical substance for the first time.

Example Ex3: The method according to Ex1 or Ex2, comprising the step of:
setting a maximum life span of the sensing module as the earliest of:
   the expiry of a predetermined time interval;
   sending the data representative of the sensed specific chemical substance for the first time.

Example Ex4: The method according to any of Ex1-Ex3, comprising:
providing the sensing module with an energy source.

Example Ex5: The method according to any of Ex1-Ex4, wherein the step of providing a substrate layer includes:
providing a flexible substrate layer.

Example Ex6: The method according to Ex4 and Ex5, comprising the step of:
generating energy by mechanical deformation of the flexible substrate layer in order to activate the wireless transmitter.

Example Ex7: The method according to Ex6, comprising:
supplying the generated energy to the wireless transmitter.

Example Ex8: The method according to one or more of Ex1- Ex7, wherein the step of providing a sensing layers includes:
covering the sensing layer with a protective layer;
   and wherein the method includes:
removing the protective layer from the sensing layer before the step of sensing the specific chemical substance in the environment.

Example Ex9: The method according to one or more of Ex1-Ex8, comprising:
setting a first threshold of the specific chemical substance; or
modify a pre-set first threshold of the specific chemical substance in the sensing layer.

Example Ex10: The method according to one or more of Ex1-Ex9, comprising:
providing the wireless transmitter with a non-rechargeable energy storage unit;
at least partially depleting the non-rechargeable energy storage unit by sending data representative of the sensed specific chemical substance to the receiving device using the wireless transmitter.

Example Ex11: The method according to one or more of Ex1-Ex10, including:
generating a warning signal when the data representative of the sensed specific chemical substance are received by the receiving device.

Example Ex12: The method according to one or more of Ex1-Ex11, comprising:
providing a set of sensing layers, each sensing layer of the set adapted to sense a specific chemical substance different from the specific chemical substance sensed by another sensing layer of the set;
selecting a predetermined specific chemical substance to be sensed; and
selecting from the set of sensing layers, a sensing layer adapted to sense the the selected chemical substance.

Example Ex13: The method according to Ex12, wherein the step of providing a set of sensing layers includes:
providing each sensing layer of the set with a machine readable code identifying the specific chemical substance sensed by the sensing layer.

Example Ex14: The method according to Ex13, including the step of:
scanning the machine readable code by means of the receiving device.

Example Ex15: The method according to one or more of Ex12-Ex14, comprising:
selecting at least two sensing layers from the set of sensing layers;
attaching the at least two sensing layers to the same substrate layer.

Example Ex16: A sensing module for detecting a specific chemical substance in an environment, comprising:
a sensing layer adapted to sense the specific chemical substance;
a substrate layer attached to the sensing layer, the substrate layer comprising:
   a wireless transmitter;
   a non-rechargeable energy storage unit electrically connectable to the wireless transmitter and adapted to contain an amount of energy sufficient to allow a single activation of the wireless transmitter;
the sensing module also comprising a switch adapted to activate the wireless transmitter by connecting it to the non-rechargeable energy storage unit if the amount of the specific chemical substance sensed by the sensing layer is above or below a first threshold, so that the wireless transmitter sends data representative of the sensed specific chemical substance.

Example Ex16: Sensing module according to Ex15, wherein the wireless transmitter comprises a transponder.

Example Ex17: Sensing module according to Ex15 or Ex16, wherein the substrate layer comprises an activation layer including the wireless transmitter, and an energy layer comprising the non-rechargeable energy storage unit, the activation layer and energy layer being attached one to the other and electrically connected.

Example Ex18: Sensing module according to one or more of Ex15-Ex17, wherein the non-rechargeable energy storage unit comprises a capacitor to store energy.

Example Ex19: Sensing module according to one or more of Ex15-Ex18, wherein the sensing layer comprises:
a protective layer covering the sensing layer.

Example Ex20: Sensing module according to one or more of Ex15-Ex19, wherein the substrate layer comprises an energy source.

Example Ex21: Sensing module according to one or more of Ex15-Ex20, wherein the energy source comprises a piezoelectric device.

Example Ex22: A kit for detecting chemical substance in an environment, comprising:
a set of sensing layers attached to a common base, each sensing layer of the set adapted to sense a specific chemical substance;
a substrate layer, the substrate layer being adapted to be attached to at least one of the sensing layers of the set so as to form a sensing module, the substrate layer comprising:
   a wireless transmitter;
   a non-rechargeable energy storage unit electrically connectable to the wireless transmitter and adapted to contain an amount of energy sufficient to allow a single activation of the wireless transmitter;
the sensing module also comprising a switch adapted to activate the wireless transmitter by connecting it to the non-rechargeable energy storage unit if the amount of the specific chemical substance sensed by the sensing layer is above or below a first threshold, so that the wireless transmitter sends data representative of the sensed amount of the specific chemical substance; and
a receiving device adapted to receive the data sent by the wireless transmitter.

Example Ex23: Kit according to Ex22, wherein the wireless transmitter comprises a transponder.

Example Ex24: Kit according to Ex22 or Ex23, wherein the substrate layer comprises an activation layer including the wireless transmitter, and an energy layer comprising the non-rechargeable energy storage unit, the activation layer and energy layer being attached one to the other and electrically connected.

Example Ex25: Kit according to one or more of Ex22-Ex24, wherein the non-rechargeable energy storage unit comprises a capacitor to store energy.

Example Ex26: Kit according to one or more of Ex22-Ex25, wherein the sensing layer comprises:
a protective layer covering the sensing layer.

Example Ex27: Kit according to one or more of Ex22-Ex26, wherein the substrate layer comprises an energy source.

Example Ex28: Kit according to one or more of Ex22-Ex27, wherein the energy source comprises a piezoelectric device.

Example Ex29: Kit according to one or more of Ex22-Ex28, wherein each sensing layer of the set comprises a machine readable indicia including information on the specific chemical substance sensed by the sensing layer.

Examples will now be further described with reference to the figures in which:

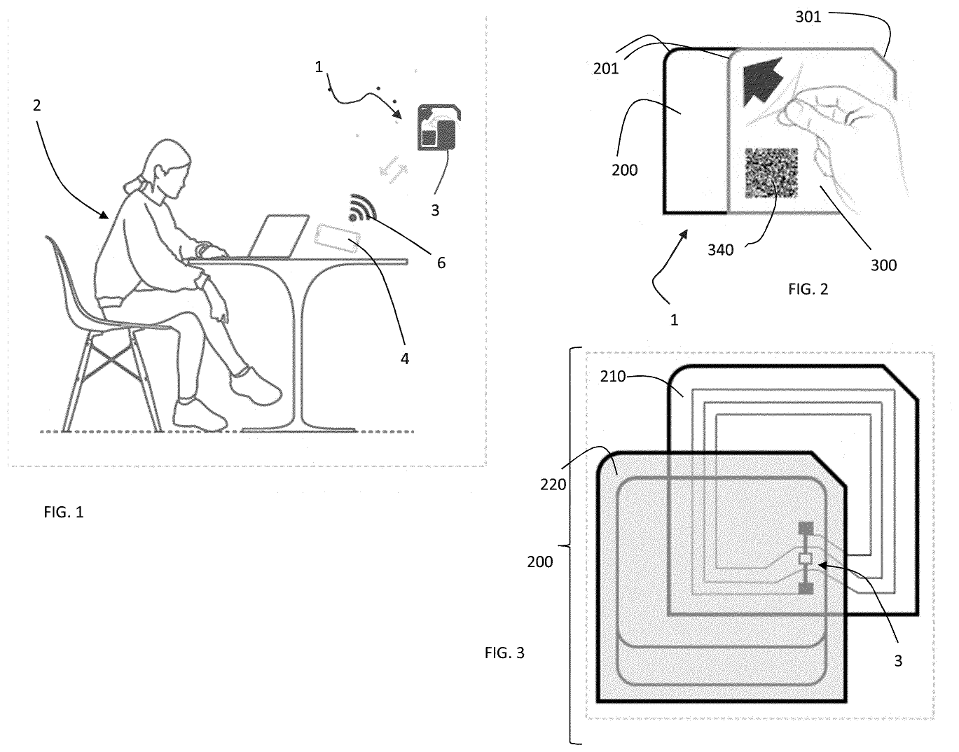

FIG. 1 is a schematic view of a possible use of the sensing module of the invention;

FIG. 2 is a schematic top view of the sensing module of the invention in a partially disassembled configuration;

FIG. 3 is a more detailed view of the sensing module of FIG. 3;

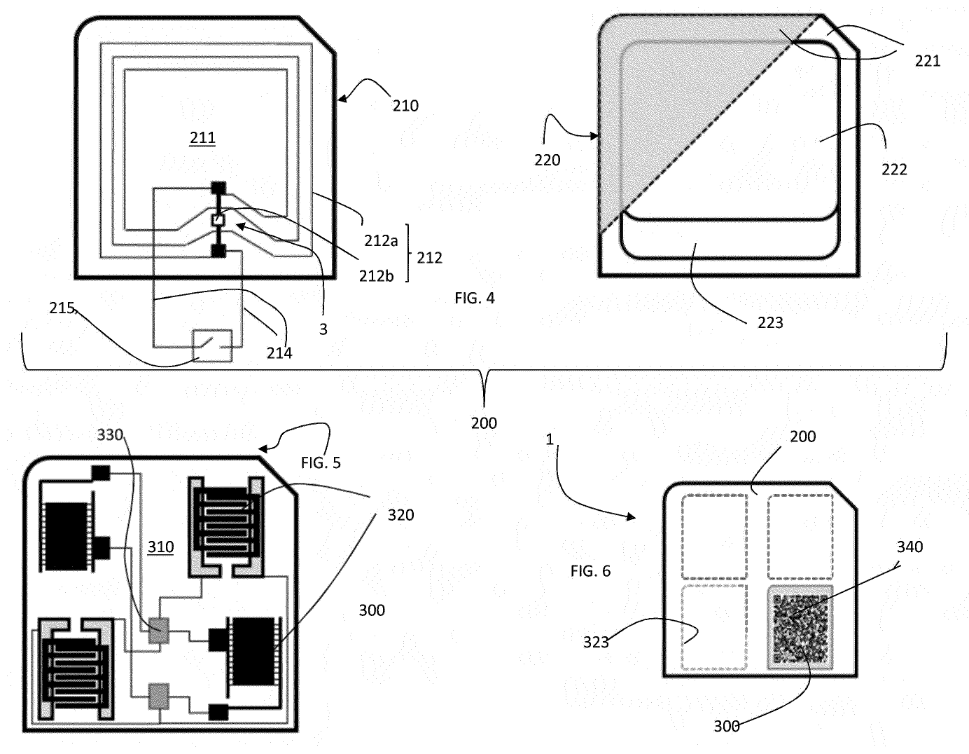

FIG. 4 is a top view of a portion of the sensing module of FIGS. 2 and 3;

FIG. 5 is a top view of a different portion of the sensing module of FIGS. 2 and 3;

FIG. 6 is a top view of a different embodiment of a sensing module of the invention;

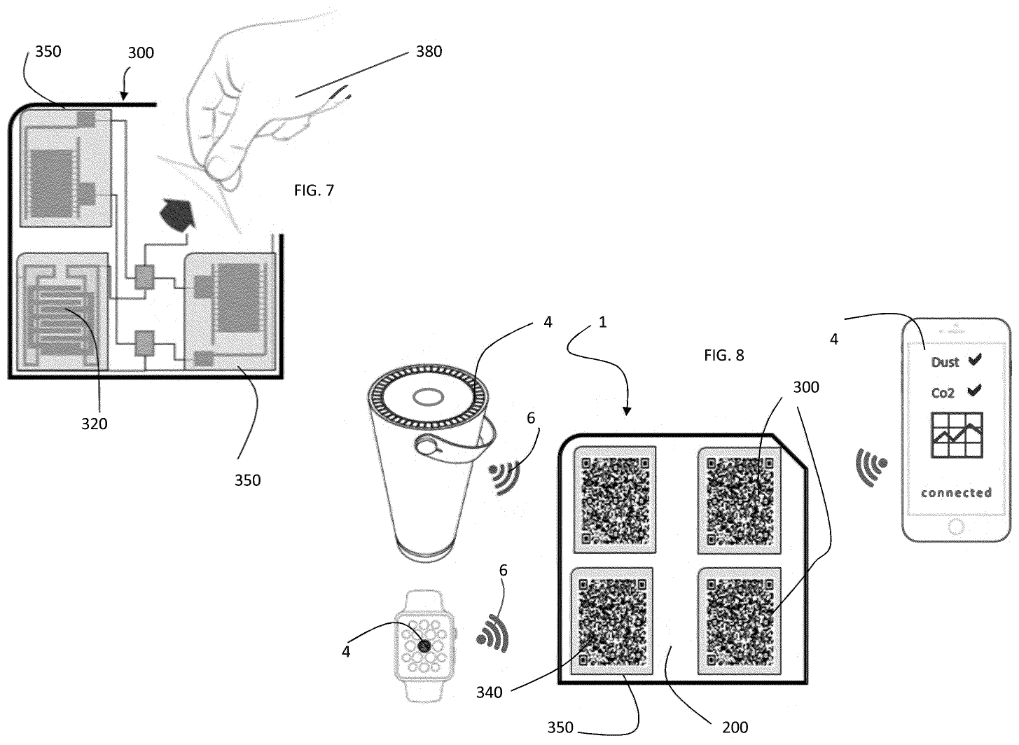

FIG. 7 is a top view of the sensing module of FIG. 6 in a different configuration;

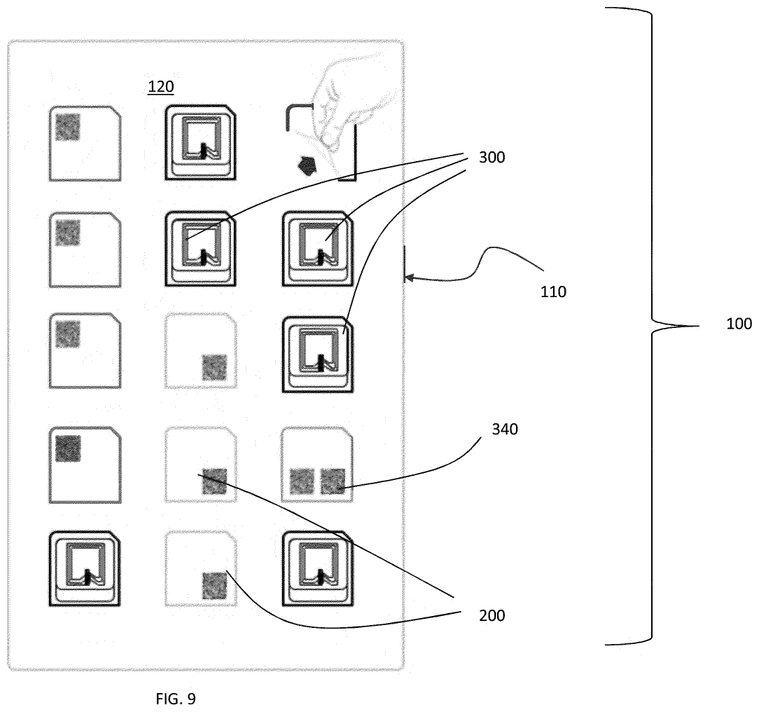

FIG. 8 is a top view of the sensing module of FIGS. 6 and 7 operating according to the method of the invention; and

FIG. 9 is a kit realized according to the invention.

A sensing module to detect the presence of a chemical substance in an environment in depicted in FIG. 1 and indicated with 1. The sensing module 1 may be used for check-up quality of environmental air indoor and outdoor around a user 2 in a flexible manner.

For that purpose, the sensing module 1 can be stick on any item, surface or object. The sticking may take place by an adhesive bond, Velcro or Blu-Tack fasteners or others (not depicted in the drawings). The sensing module include a wireless transmitter 3. Preferably the sensing module 1 is integrated into a personal area network (PAN). By the PAN or any other network, the sensing module 1 may be connected to a smart device 4 like a smartphone, smartwatch or smart air purifier which can alert the user 2, if the chemical substance is present in the surrounding of the sensing module, or it is present above or below a pre-set threshold.

The sensing module 1 is a single-use sensing module 1 as detailed below.

With now reference to FIG. 2, the sensing module 1 comprises a substrate layer 200 and at least one sensing layer 300. The substrate layer 200 and the sensing layer 300 are sheet-like and preferably have the same dimensions and geometrical shape. Preferably, on a top view, the substrate layer 200 and the sensing layer 300 are substantially rectangular. Preferably, the substrate layer 200 and the sensing layer 300 are attached to each other, so that the substrate layer adheres on top of the substrate layer, or vice-versa. Preferably, their dimensions are 40 millimetres×40 millimetres×3 millimetres, where the smallest dimension is their thickness.

The substrate layer 200 may include an adhesive layer (not depicted in the drawings) on one of its surfaces, the surface opposite to the one attached to the sensing layer 300, to adhere to any item, surface or object. The sensing layer 300 may also include an adhesive layer on one of its surfaces, to adhere to the substrate layer 200. The connection between substrate layer 200 and sensing layer 300 is such that an electrically conductive connection is established between the two layers.

As visible in FIG. 2 where the two layers 200, 300 forming the sensing module 1 are detached from each other for clarity purposes, both the sensing layer 300 and substrate layer 200 includes a “rounded” corner 201 and a “cut” corner 301 which predefine in which manner the sensing layer and the substrate layer have to be stick together. By matching the same corners 201 and 301 in both layers 200, 300, the user can get sure to attach the substrate layer 200 and the sensing layer 300 accurately together.

With now reference to FIG. 3, the substrate layer 200 comprises an activation layer 210 and an energy layer 220. The activation layer 210 and the energy layer 220 are attached to each other in such a way that an electrical connection between the two layers 210, 220 is present. The activation layer 210 and energy layer 220 are one on top of the other.

Activation layer 210 and energy layer 220 are depicted in more detail in FIG. 4. The activation layer 210 comprises a flexible plastic substrate 211. On the flexible plastic substrate 211, the wireless transmitter 3 is formed. The wireless transmitter 3 may include a transponder 212. The transponder comprises a coupling antenna 212a and a transponder chip 212b. The transponder 212 is connected via wires 214 to a switch 215.

The energy layer 220 comprises an energy source. The energy source includes a piezoelectric nanocomposite layer 222. The piezoelectric nanocomposite layer 222 may be formed by a layer made of composite of BaTiO₃, ZnO, MoS₂ or WSe₂ located between two flexible metal-coated plastic substrates 221. By this configuration, the energy layer 220 is able to generate electrical energy from mechanical deformation. In the process of sticking the sensing module 1 on any item, surface or object, the energy module 1, and in turn the energy layer 220, is possibly bent and electric energy can be generated. In order to store the energy generated by the mechanical deformation, the energy layer 220 further comprises a flexible capacitor 223. The flexible capacitor 223 preferably comprises a layer of nylon films or polyethene naphthalate substrates with ZrO₂ as a dielectric layer. Thereby, the energy layer 220 can generally generate an output of about 1.5-2 Volts and 150-250 nanoAmperes.

As illustrated in FIG. 5, the sensing layer 300 includes a transparent and flexible plastic substrate 310 and a sensor 320 adapted to measure or detect a chemical substance. The sensor 320 is generally applied to the substrate 310 by ink printing, sputtering or vapour disposition. Preferably, several sensors 320 may be applied to the substrate 310 depending on the given amount of space on the sensing layer 300.

In the embodiment depicted in FIG. 6, the sensing module 1 comprises several sensing layers 300 attached to the same substrate layer 200. Preferably, each sensing layer 300 is adapted to sense a different chemical substance. In the depicted embodiment, the substrate layer 200 may include four sensing layers 300. For example, the sensing layers 300 may be attached one on top of the other, but each sensing layer 300 may include a cut-out 323 in the substrate 310, to ensure that the sensors 320 of the layers underneath do not get covered by the substrate 310 of the overlapping sensing layers 300 and as a result all sensors 320 can measure and monitor different chemical substances properly. For example in FIG. 6, four different sensing layers 300 may be present, which can detect four different chemical substances, such as four different pollutants.

Preferably, to each sensor 320 a threshold is associated. The threshold refers to the measured chemical substance by the sensor.

The sensors 320 present in the sensing layer 300 may be for example carbon nanotube sensors or metal oxide sensors. Sensors for different chemical substances can also be applied to the same substrate 310 of the same sensing layer 300, whereby each individual sensor can be responsible for a specific pollutant and a corresponding threshold level.

The sensors 320 are electrically interconnected, for example by an OR-circuit, with the switch 215 of the substrate layer 200 and a contact area 330 of the sensing layer 300.

Other sensors (not depicted in the drawings) may be located on the substrate 310 of the sensing layer 300, like biosensors or sensors responsive to UV light, temperature, provided that integration into the flexible plastic substrate 310 is given.

As shown in FIG. 7, the sensors 320 of the sensing layer 300 may be sealed by a protective film 350. Thereby, sensors 320 may be protected by the protective film 350.

Furthermore, when the sensors 320 are covered by the protective film 350, measurements of the chemical substance are not possible. Thus, the sensing module 1 is secured from unintended measuring and may only monitor the chemical substance(s) when the protective film 350 is removed.

Each sensing layer 300 may include a QR code 340 (see FIGS. 6 and 8), which may for example be imprinted on the protective film 350. Information of the specifications of the sensors 320 and the type of chemical substance detected by the sensors may be included in the QR code 340. In an alternative embodiment, the QR code 340 is imprinted on another scannable area of the sensing module 1.

The sensing module 1 may be formed using a kit 100, as depicted in FIG. 9.

The kit 100 may include a sticker set 110 which includes on the same substrate 120 a plurality of sensing layers 300. The sticker set, in the same substrate 120, also comprises a plurality of substrate layers 200. As shown in FIG. 9, all layers 200,300 are thereby stuck on substrate 120, for example bonding paper.

Each sensing layer 300 preferably includes a QR code 340 to indicate the chemical substance that is adapted to sense. A user may create their individual sensing module 1 adapted to their specific situation.

In that way, a user may remove one or more chosen sensing layers 300 and stick them on top of a base layer 200, also removed from the substrate 120. Several sensing layers 300 can be stuck one above the other on top of a substrate layer 200. In order to select the desired sensing layers 300 from the sticker set 110, the user may scan the QR codes 340 to detect the desired sensors 320 for the chemical substance of interest.

A sensing module 1 created from the kit 100 including four different sensing layers 300 is represented in FIG. 8.

The functioning of the sensing module 1 is as follows.

The sensing module 1 is either already provided, or it is created by the user by selecting the suitable sensing layers 300 from the kit 100. The selected sensing layers 300 are then attached to the substrate layer 200.

In order to activate the sensor module 1, the protective film 350 is removed.

The sensor module 1 may need to be configured. The configuration may be performed using the smart device 4 which read the QR code 340. The smart device may for example set the threshold for each sensor 320 present in the sensing module 1.

After the configuration of the sensing module 1, a user may choose any environment where they want to monitor the air in order to detect the presence of certain chemical substance (those measured by the selected sensing layers) and stick the sensor module 1 on any item, surface or object. The smart device 4 is adapted to receive a signal 6 (depicted schematically in FIGS. 1 and 8) sent by the sensing module 1.

By pulling off the protective film 350, the sensing module 1 is activated and the sensors 320 start measuring the presence and amount of the selected chemical substances. If at a certain point in time a certain chemical substance, such as a pollutant, is detected to be in an amount exceeding the threshold set by the smart device 4, an alerting signal 6 is sent by the sensing module 1 to the smart device 4 of the user. This is schematically depicted in FIGS. 8 and 1. In FIG. 8, several smart devices 4 are depicted, such as a smart watch, smartphone or loudspeaker.

In order to send the signal 6 to the smart device 4, the transmitter 3 is used. When the selected threshold is reached for any chemical substance as measured by the sensor 320, the switch 215 closes. When the switch 215 is closed, the energy layer 220 feeds the activation layer 210 with the energy stored in the capacitor 223 which has been previously charged by the energy source. The energy coming from the capacitor 223 is enough to activate the antenna 212a of the transponder 212 and thus to send the alerting signal 6.

Thereby, the sensing module 1 does not dependent on any external power supply, does not have to be charged and can, therefore, be used in a more flexible manner.

Due to the fact that the capacitor 223 is not rechargeable, the sensing module 1, after the signal has been sent by the antenna 212a, is not reusable. The sensing module is thus preferably discarded. The sensing module 1 is indeed single use and can be used up to, and including, the moment in which the signal is sent to the smart device 4.

As the effects and impacts of allergic and respiratory reactions vary from person to person, the threshold level of a monitored air pollutant can be adapted by the user to his specific requirements via the connected smart device. Thereby the sensing module is set up for single-use and has fulfilled its function after sending an alerting signal to a user if a harmful (for the user) chemical substance is detected. As a result, users can avoid forthcoming allergic reactions or further health consequences of air pollution.

For the purpose of the present description and of the appended claims, except where otherwise indicated, all numbers expressing amounts, quantities, percentages, and so forth, are to be understood as being modified in all instances by the term “about”. Also, all ranges include the maximum and minimum points disclosed and include any intermediate ranges therein, which may or may not be specifically enumerated herein. In this context, therefore, a number A is understood as A±10 percent of A. Within this context, a number A may be considered to include numerical values that are within general standard error for the measurement of the property that the number A modifies. The number A, in some instances as used in the appended claims, may deviate by the percentages enumerated above provided that the amount by which A deviates does not materially affect the basic and novel characteristic(s) of the claimed invention. Also, all ranges include the maximum and minimum points disclosed and include any intermediate ranges therein, which may or may not be specifically enumerated herein.

1. A method to detect a specific chemical substance in an environment, the method comprising:
providing a sensing layer adapted to sense the specific chemical substance;
providing a substrate layer, the substrate layer comprising a single-use wireless transmitter designed to be used only once and a non-rechargeable energy storage unit electrically connectable to the wireless transmitter and adapted to contain an amount of energy sufficient to allow a single activation of the wireless transmitter;
attaching the sensing layer to the substrate layer so as to form a sensing module;
sensing the amount of the specific chemical substance in the environment by means of the sensing layer; and
if the amount of the specific chemical substance is above or below a first threshold, sending data representative of the sensed specific chemical substance to a receiving device using the wireless transmitter.

2. The method according to claim 1, comprising the step of:
discarding the sensing module after sending the data representative of the sensed specific chemical substance for the first time.

3. The method according to claim 1, comprising the step of:
setting a maximum life span of the sensing module as the earliest of:
the expiry of a predetermined time interval;
sending the data representative of the sensed specific chemical substance for the first time.

4. The method according to claim 1, comprising:
providing the sensing module with an energy source.

5. The method according to claim 1, wherein the step of providing a substrate layer includes:
providing a flexible substrate layer.

6. The method according to claim 5, comprising the step of:
generating energy by mechanical deformation of the flexible substrate layer in order to activate the wireless transmitter.

7. The method according to claim 1, wherein the step of providing a sensing layers includes:
covering the sensing layer with a protective layer;
and wherein the method includes:
removing the protective layer from the sensing layer before the step of sensing the specific chemical substance in the environment.

8. The method according to claim 1, comprising:
setting a first threshold of the specific chemical substance; or
modify a pre-set first threshold of the specific chemical substance in the sensing layer.

9. The method according to claim 1, comprising:
providing the wireless transmitter with a non-rechargeable energy storage unit;
at least partially depleting the non-rechargeable energy storage unit by sending data representative of the sensed specific chemical substance to the receiving device using the wireless transmitter.

10. The method according to claim 1, including:
generating a warning signal when the data representative of the sensed specific chemical substance are received by the receiving device.

11. The method according to claim 1, comprising:
providing a set of sensing layers, each sensing layer of the set adapted to sense a specific chemical substance different from the specific chemical substance sensed by another sensing layer of the set;
selecting a predetermined specific chemical substance to be sensed;
selecting from the set of sensing layers, a sensing layer adapted to sense the selected chemical substance.

12. The method according to claim 11, comprising:
selecting at least two sensing layers from the set of sensing layers;
attaching the at least two sensing layers to the same substrate layer.

13. A sensing module for detecting a specific chemical substance in an environment, comprising:
a sensing layer adapted to sense the specific chemical substance;
a substrate layer attached to the sensing layer, the substrate layer comprising:
a wireless transmitter designed to be used only once;
a non-rechargeable energy storage unit electrically connectable to the wireless transmitter and adapted to contain an amount of energy sufficient to allow a single activation of the wireless transmitter;
the sensing module also comprising a switch adapted to activate the wireless transmitter by connecting it to the non-rechargeable energy storage unit if the amount of the specific chemical substance sensed by the sensing layer is above or below a first threshold, so that the wireless transmitter sends data representative of the sensed specific chemical substance.

14. A kit for detecting chemical substance in an environment, comprising:
a set of sensing layers attached to a common base, each sensing layer of the set adapted to sense a specific chemical substance;
a substrate layer, the substrate layer being adapted to be attached to at least one of the sensing layers of the set so as to form a sensing module, the substrate layer comprising:
a wireless transmitter designed to be used only once;
a non-rechargeable energy storage unit electrically connectable to the wireless transmitter and adapted to contain an amount of energy sufficient to allow a single activation of the wireless transmitter;
the sensing module also comprising a switch adapted to activate the wireless transmitter by connecting it to the non-rechargeable energy storage unit if the amount of the specific chemical substance sensed by the sensing layer is above or below a first threshold, so that the wireless transmitter sends data representative of the sensed specific chemical substance; and
a receiving device adapted to receive the data sent by the wireless transmitter.

15. The sensing module according to claim 13, wherein the substrate layer comprises an activation layer including the wireless transmitter, and an energy layer comprising the non-rechargeable energy storage unit, the activation layer and energy layer being attached one to the other and electrically connected.

16. The kit according to claim 14, wherein the substrate layer comprises an activation layer including the wireless transmitter, and an energy layer comprising the non-rechargeable energy storage unit, the activation layer and energy layer being attached one to the other and electrically connected.
